(19) United States
(12) Patent Application Publication
(10) Pub. No.: US 2009/0068749 A1
(43) Pub. Date: Mar. 12, 2009

(54) MODULE FOR AUTOMATED MATRIX REMOVAL IN ACIDIC PLATING SOLUTIONS

(76) Inventor: Harmesh K. Saini, Santa Clara, CA (US)

Correspondence Address: MACPHERSON KWOK CHEN & HEID LLP, 2033 GATEWAY PLACE, SUITE 400, SAN JOSE, CA, 95110, US

(21) Appl. No.: 12/205,617

(22) Filed: Sep. 5, 2008

Related U.S. Application Data
(63) Continuation of application No. PCT/US2007/063425, filed on Mar. 6, 2007.
(60) Provisional application No. 60/780,402, filed on Mar. 7, 2006.

(51) Int. Cl. G01N 33/20; B01J 19/00

(52) U.S. Cl. 436/76; 422/68.1

(57) ABSTRACT

In accordance with the present invention, an organic additive is characterized in the presence of an acidic metal plating matrix in a metal plating solution by: providing a sample from the metal plating solution; activating a metal-complexing resin with a weak acid to provide an activated metal-complexing resin; eluting the sample through the activated metal-complexing resin to form a treated sample in which a concentration of the acidic metal plating matrix is reduced; and determining a concentration of an organic additive in the metal plating solution by analyzing the treated sample.

### RELATED APPLICATION

This application is a Continuation of International Application No. PCT/US2007/63425, filed Mar. 6, 2007, which in turn claims the benefit of U.S. Provisional Application No. 60/780,402, filed Mar. 7, 2006, the contents of both applications being incorporated by reference in their entirety.

### TECHNICAL FIELD

The present invention relates generally to chemical analysis, and more particularly to apparatus for the removal of chemical interferents prior to chemical analysis.

### BACKGROUND

Automated systems for measuring the concentration of analytes in a sample have been developed using a number of analytical techniques such as chromatography or mass spectrometry. For example, co-assigned U.S. patent application Ser. No. 10/094,394, entitled “A Method and Apparatus for Automated Analysis and Characterization of Chemical Constituents of Process Solutions,” filed Mar. 8, 2002, the contents of which are hereby incorporated by reference in their entirety, discloses an automated in-process mass spectrometry (IPMS) apparatus for identifying and quantifying chemical constituents and their reaction products in process solutions. One type of process solution which the IPMS apparatus in the above-mentioned application may analyze is a copper electroplating bath for the deposition of copper structures on semiconductor wafers. The bath comprises a relatively concentrated acidic aqueous copper sulfate solution. Plating topology is controlled by organic plating solution additives within the copper sulfate solution that function to regulate, suppress, or accelerate the plating process. These additives experience electrochemical breakdown during the plating process and can be lost by drag out or by becoming trapped within the copper plating film. But the achievement of void-free plating in the vias and trenches of sub-micron high-aspect-ratio structures requires very tight control of additive levels. Unlike indirect measurement methods such as cyclic voltametric stripping (CVS) that monitor the effectiveness of the plating solution, the IPMS apparatus discussed above allows a user to directly measure the additive concentration plus the breakdown products in the electroplating bath to ensure a defect-free deposition process.

High sensitivity quantification of the organic additives and their breakdown by-products in the electroplating bath is hampered by the relatively high concentration of sulfuric acid and copper sulfate matrix within the bath. These relatively high concentrations of sulfuric acid, sulfate, and copper ions obscure the detection and quantification of the organic additive ions because ionization of the higher concentration ions is statistically more likely in the ionization source of the mass spectrometer. Thus, the copper sulfate should be removed from the sample and/or the pH adjusted to quantify the organic additive concentration. Similarly, other metrology techniques such as flow injection analysis and chromatography often require the removal of chemical constituents that may hamper the detection or quantification of an analyte of interest.

To address the need in the art for automated systems that remove chemical interferents, U.S. application Ser. No. 11/254,030, filed Oct. 18, 2005, discloses a matrix removal module that is configurable to automatically receive a sample containing an interferent. A precipitating reagent is also introduced into the module to mix with the received sample. The precipitating reagent forms a precipitant through reaction with the chemical interferent. The module filters the precipitant and then flushes the corresponding filter to remove the precipitant. For example, the received sample may be a solution of copper sulfate and the precipitating reagent may be a solution of Ba(OH)₂ such that the precipitate is Cu(OH)₂. Although such a module offers an automated removal of the chemical interferent, the process can be sensitive to the specific concentrations of copper sulfate and Ba(OH)₂. For example, if the Ba(OH)₂ solution is not concentrated enough, the treated solution will still contain copper sulfate and sulfuric acid. On the other hand, if the Ba(OH)₂ solution is too concentrated, the treated solution may be undesirably basic.

To provide the appropriate amount of reagent, the concentration of the matrix may be assumed to be static such that a fixed volume of reagent solution may be added based upon the volume of sample solution needing matrix elimination. Alternatively, the matrix concentration may be measured periodically such that the volume of reagent solution being added depends upon the latest measured matrix concentration. For example, it has been shown that the copper sulfate concentration and sulfuric acid concentration in copper electroplating bath solution has appreciable variability in semiconductor manufacturing operations. Thus, an addition of a fixed volume of Ba(OH)₂ solution to samples of copper electroplating solutions may result in either under-precipitation or over-precipitation of the copper sulfate/sulfuric acid concentrations. In general, an addition of a variable volume of reagent solution based upon periodic measurements of the concentrations of the matrix avoids such over-precipitation or under-precipitation problems. Such embodiments may be denoted as “closed loop” embodiments in that feedback information (the latest measurement of the matrix concentration to be eliminated) is used to update the amount of reagent being added. In contrast, embodiments in which the amount of reagent being added is static may be denoted as “open loop” embodiments in that no feedback information is utilized.

Regardless of whether a fixed volume or variable volume of reagent solution is used, the use of a precipitating reagent such as Ba(OH)₂ may be problematic in certain situations due to environmental concerns (Ba(OH)₂ being quite toxic). Moreover, the need for feedback information regarding the matrix concentration for applications such as copper plating solution monitoring complicates the design should the treated sample need to have a tightly-controlled pH. In addition, the use of Ba(OH)₂ may precipitate organic additives of interest, thereby leading to inaccurate concentration measurements.

Accordingly, there is a need in the art for improved automated systems for the removal of chemical interferents prior to a chemical analysis that do not require feedback information.

### SUMMARY

In accordance with an aspect of the present invention, a method of analyzing a metal plating solution is provided. The method includes: activating a metal-complexing resin with a weak acid to provide an activated metal-complexing resin;

eluting a sample of metal plating solution through the activated metal-complexing resin to form a sample of treated metal plating solution; and determining a concentration of an organic additive in the treated metal plating solution.

In accordance with another aspect of the invention, an analytical apparatus is provided that includes: a sample extraction module operable to extract a sample of known volume from a metal plating solution having an acidic metal plating matrix; a sample dilution module operable to dilute the sample to provide a diluted sample; a mixer operable to mix the diluted sample with a spike to form a spiked sample, a column packed with metal-complexing resin to reduce a concentration of the acidid metal plating matrix in the spiked sample to provide a treated sample; an atmospheric pressure ionizer operable to ionize the treated sample to produce ions; a mass spectrometer operable to process the ions to provide a ratio response; and a control system operable to control a cyclic extraction of samples, dilution of the samples, spiking of the diluted samples, treatment of the spiked diluted samples, ionization of the treated samples, processing of the ions to provide ratio responses, and processing of the ratio responses to characterize the concentration of an organic additive in the metal plating solution over time.

### BRIEF DESCRIPTION OF THE DRAWINGS

FIG. 1 is a schematic illustration of a matrix removal system including a weak anion exchange column according to an embodiment of the invention.

FIG. 2 is a schematic illustration of a matrix removal system that does not include a weak anion exchange column according to an embodiment of the invention.

FIG. 3 illustrates an in-process-mass-spectrometry (IPMS) system that includes a matrix removal system in accordance with an embodiment of the invention.

### DETAILED DESCRIPTION

A matrix elimination module is disclosed that exploits the reversible nature of certain chelating agents such as bis-picoylamine. Acidic plating solutions are first treated with an activated chelating resin to remove metals from the solution. The treated solution may then have its remaining acidic matrix removed using a weak anion exchange resin. Alternatively, the chelating resin may be activated with a weak acid such that basic sites within the chelating resin are still available to combine with protons in the acidic matrix, thereby reducing the acidity of the matrix. In this fashion, an acidic plating matrix such as copper sulfate may be removed using activated chelated resin without requiring any additional components such as a weak anion exchange resin.

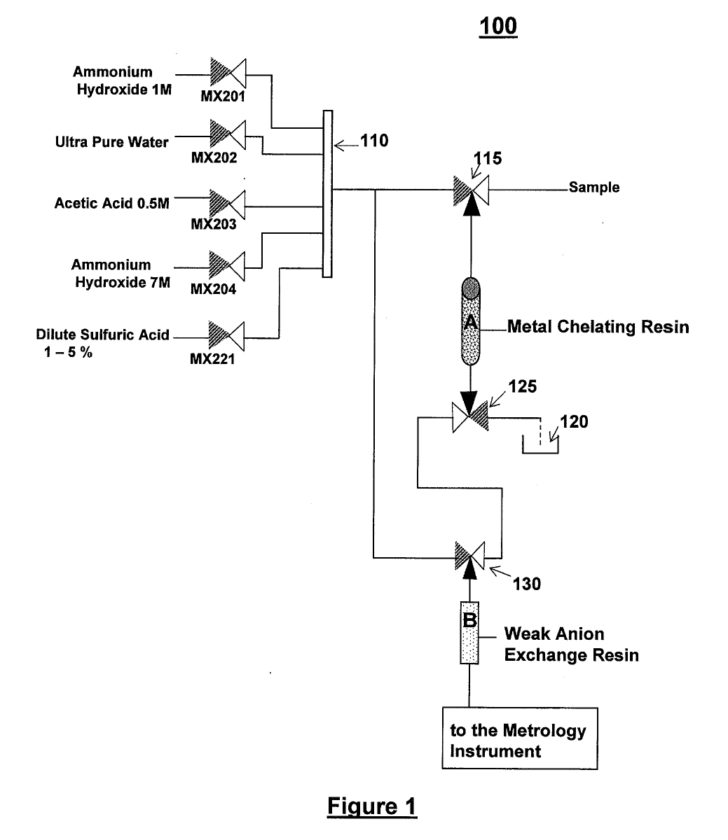

Turning now to the drawings, FIG. 1 illustrates a block diagram of an automated system 100 for removing the matrix of an acidic plating solution. A column A is packed with a chelating resin having basic sites that may reversibly remove metals from acidic solutions. For example, column A may be packed with Dowex M-4195 chelating resin manufactured by Dow Chemical Company. The functional group in this resin is bis-picoylamine that is activated by exposure to a dilute acid such as 1 to 5% sulfuric acid. This acid activation causes the bis-picoylamine to partially quaternize into a sulfuric acid salt form that is then ready to scavenge metals from acidic solutions. Activated bis-picolyamine has an enhanced affinity for copper and is thus ideal for scavenging copper from acidic copper plating solutions. The following discussion will thus describe an embodiment using an activated bis-picoylamine-containing resin. However, other chelating resins adapted to scavenge metals in acidic solutions may also be used to pack column A. Ideally, the chelating resin is amenable to regeneration but single use resins may also be used.

To activate the bis-picoylamine functional groups in the resin, a dilute sulfuric acid solution (1 to 5%) may be selected for at a manifold 110 through activation of a valve MX221. A controller (not illustrated) such as discussed in U.S. application Ser. No. 11/298,738, entitled “In-Process Mass Spectrometry With Sample Multiplexing,” (the “sample multiplexing application”), filed Dec. 9, 2005, the contents of which are incorporated by reference herein, may be used to control the activation of the various components in system 100. The dilute sulfuric acid may then flow through a three-way valve 115 into column A to activate the resin. After passing through column A and a three-way valve 125, the dilute sulfuric acid may pass into a drain 120.

If desired, column A may then be rinsed with ultra-pure water (UPW) through activation of a valve MX202 at manifold 110. Having been activated, column A is then ready to receive a sample of acidic plating solution such as copper plating solution through appropriate activation of valve 115. The eluent passing from column A will then be substantially free of metals such as copper.

This eluent may then flow through valve 125 and a valve 130 into a column B packed with weak anion exchange resin. In general, an ion exchange resin such as a weak anion exchange resin is an organic polymer to which active groups have been covalently attached. Depending on the properties of these groups, an ion exchange resin may be classified as either a cation or anion exchange resin. In an anion exchange resin, the functional or active groups that have been covalently bonded to the resin backbone are positively charged so that they may exchange negatively charged counter ions (anions). An anion exchange resin may be classified as either a weak or strong anion exchange resin depending upon the basicity of the active groups. As suggested by the name, the active groups in a weak anion exchange resin are weakly (rather than strongly) basic. Generally, a weak anion exchange resin uses tertiary amines or polyamines as the functional groups but it will be appreciated that numerous other functional or active groups having a sufficiently weak basicity (and suitability for covalent bonding to the resin) may also be used.

Should an analysis require a substantially neutral pH, the weak anion exchange resin may be merely regenerated prior to treating the eluent from column A. However, certain analyses require a more acidic pH. For example, the analysis of organic leveler additive in a semiconductor copper plating bath solution is preferentially performed at a relatively acidic pH such as between a pH of 4 and 5. Outside of this pH range, the accuracy of the analysis may suffer. Advantageously, the acidic eluent from column A (having its copper scavenged but still containing sulfuric acid) may be brought into the desired pH range by eluting this acidic solution through column B after the weak anion exchange resin has been activated with a suitable weak acid such as dilute acetic acid (0.5M). To activate column B, the acetic acid solution may be selected for at manifold 110 through activation of a valve MX203. The acetic acid may then flow through valve 130 into column B and then into a drain (not illustrated).

As used herein, a “weak acid” has a pKa whose relationship to the pKa for the functional groups in the weak anion exchange resin is such that a substantial portion of the functional groups are left un-protonated after exposure to the weak acid. In turn, because of the relationship of the pKa for the sulfuric acid eluting from column A (which is a relatively strong acid) to that of the weak acid such as 0.5M acetic acid used to activate column B, the eluent from column B will still be slightly acidic and kept reliably in the pH range of between 4 and 5.

Advantageously, even though the concentration of the sulfuric acid in the plating solution being sampled will vary over time, no “feedback” operation of system 100 is necessary analogous to that discussed for the matrix elimination module disclosed in U.S. application Ser. No. 11/254,030. It will be appreciated, however, that for more effective matrix elimination, the plating solution being processed by system 100 is first diluted using a dilution module (not illustrated) such as disclosed in U.S. application Ser. No. 10/641,480, entitled “Loop Dilution System,” the contents of which are incorporated by reference. For example, such a sample dilution module may dilute the sample by a ratio 50:1 to reduce the copper ion concentration that must be adsorbed by column A and to also reduce the proton concentration with respect to adsorption in column B. The eluent from column B, having had the copper and acid matrix removed, may then be analyzed in a suitable metrology instrument (not illustrated) such as a mass spectrometer or a high performance liquid chromatography system.

After treating the sample, columns A and B may be regenerated using a solution of ammonium hydroxide at a suitable concentration. For example, column A may be regenerated with 7M ammonium hydroxide whereas column B may be regenerated using 1M ammonium hydroxide. These solutions may be selected for at manifold 110 using valves MX204 and MX201, respectively.

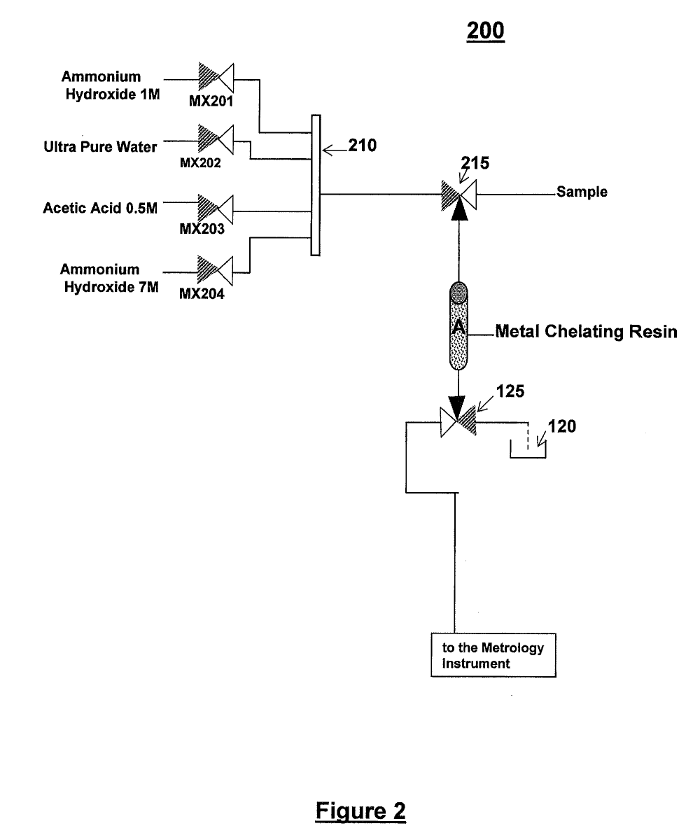

In an alternative embodiment, an acidic metal plating matrix is eliminated by a weak-acid-activated metal chelation resin. For example, each functional group in a bis-picoylamine-containing chelation resin contains three weakly basic nitrogen atoms. If this weakly basic functional group is activated with a weak acid, the relationship of the pKa for the weak acid to the pKa for the weakly basic functional group is such that a substantial portion of the nitrogen atoms in each functional group are unprotonated. However, because a remaining substantial portion of the nitrogen atoms in each functional group are protonated, a sufficient amount of bis-picoylamine is partially quaternized into a sulfuric acid salt form that is then ready to scavenge metals such as copper from an acidic metal plating solution. In this fashion, the quaternized portion of bis-picoylamine will scavenge metals while the remaining un-quaternized portion is available to reduce acidity through protonation. Turning now to FIG. 2, an automated system 200 for removing the matrix of an acidic plating solution is illustrated. A column A is packed with a metal chelating resin such as bis-picoylamine-containing chelation resin (e.g., Dowex M-4195 chelating resin). To activate this resin, a weak acid such as 0.5 M acetic acid is selected for at a manifold 210 through activation of a valve MX203. The acetic acid then flows through a three-way valve 215 into column A and into a drain 120 through a three-way valve 125 until the column has been flushed and activated. The column may be rinsed in a similar fashion by selecting for a solvent such as UPW at manifold 210 through activation of a valve MX202. The rinsed column may then receive sample through appropriate activation of valve 215. As discussed above, because the resin has been activated with a weak acid, a sufficient number of unprotonated functional groups are available to scavenge excess protons from the acidic plating matrix in the sample. Conversely, because the remaining functional groups have been acid-activated, there are a sufficient number of activated functional groups to scavenge metals from the acidic plating matrix. To ensure an appropriately-treated sample, eluent from column A may initially flow into drain 120. After an appropriate time has elapsed, the sample eluent from column A may flow to the downstream metrology instrument (not illustrated) such as a mass spectrometer or a high performance liquid chromatography system. After treating a sample, the column may be regenerated by selecting for a suitable base such as 7M ammonium hydroxide solution using a valve MX204. After regenerating the column, the ammonium hydroxide solution may flow into drain 120. After flushing with a solvent such as UPW, the column may then be re-activated with acetic acid solution, treat another sample, and so on. In this fashion, the same column may be used to cyclically treat sample after sample under machine control.

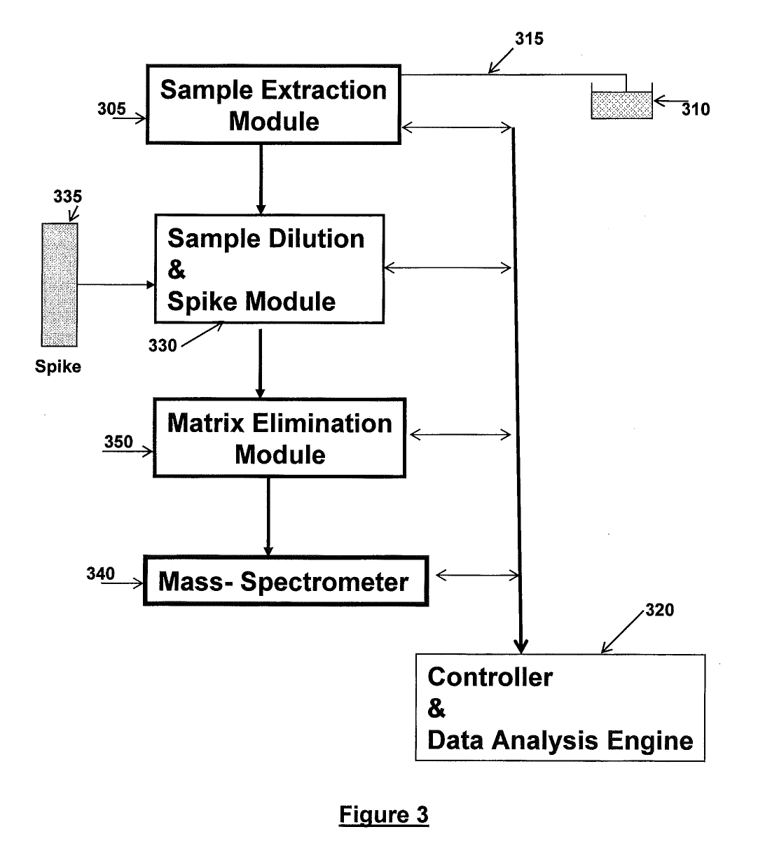

In one embodiment, a matrix elimination apparatus as disclosed herein may be incorporated into an in-process-mass-spectrometry (IMPS) system 300 as shown in FIG. 3. IPMS system 300 includes a plurality of modules. A sample extraction module 305 is configured to extract sample from one or more process solution baths 310. An exemplary sample extraction module (SEM) is disclosed in U.S. application Ser. No. 11/298,738, entitled “In-Process Mass Spectrometry with Sample Multiplexing,” the contents of which are incorporated by reference herein. As discussed in this application, SEM 305 may include a reservoir (not illustrated) having a conduit 315 connected to bath 310. Vacuum is applied to the reservoir as commanded by a controller 320. The reservoir then fills with an extracted sample. By pressurizing the reservoir (as commanded by controller 320) using a compressed gas source, the extracted sample is sent to a sample dilution and spike module 330.

An exemplary sample dilution and spike module 330 is disclosed in commonly-assigned U.S. Pat. No. 6,998,095, the contents of which are incorporated by reference in their entirety. In one embodiment of module 330, extracted sample fills a first loop or conduit attached to a first multi-way valve (not illustrated). Spike solution from a spike source 335 fills a second loop attached to this first multi-way valve. The multi-way valve may then be actuated such that the loops are connected with a diluent source such as a syringe pump containing a desired amount of diluent. The contents of the loops may then be mixed and diluted with the diluent to an appropriate dilution ratio such as 50:1. Should additional dilution be required, the diluted and spiked sample from the first multi-way valve may then be processed in additional dual-loop multi-way valves. It will be appreciated, however, that other techniques may be used to mix sample and spike solutions with appropriate diluents.

Although the matrix concentration in the resulting diluted and spiked sample from sample dilution and spike module 330 is reduced, analysis of organic additives and their breakdown products may be hampered by the relatively high concentration of matrix that remains. For example, analysis of the concentrations of organic additives and their breakdown by-products in a Cu electroplating bath is hampered by the relatively high concentration of the matrix of sulfuric acid and copper sulfate within the bath. The resulting relatively high concentrations of protons, sulfate, and copper ions obscure the detection and quantification of constituents such as organic additives because ionization of the higher concentration ions is statistically more likely in the ionization source of a mass spectrometer 340. Thus, the matrix of copper sulfate and sulfuric acid should be removed from the diluted and spiked sample from module 330 to more accurately quantify the organic additive and breakdown product concentrations.

To perform this matrix elimination, a matrix elimination module 350 such as disclosed herein with regard to FIGS. 1 and 2 processes the diluted and spiked sample from module 330. The resulting processed diluted and spiked sample is then provided by module 350 to a mass spectrometer 340. Mass spectrometer 340 may comprise a time of flight (TOF) electrospray mass spectrometer. However, it will be appreciated that other types of mass spectrometers may be implemented in the present invention such as inductively-coupled-plasma mass spectrometers.

With regard to IPMS system 300, the analysis it performs may be considered closed loop because each extracted sample that is analyzed is analyzed with regard to an added spike solution having a known volume and concentration. Such an analysis may be contrasted with an “open loop” measurement in which an extracted sample is analyzed with regard to a previously-determined calibration standard. It will thus be appreciated that the closed loop automation practiced by IPMS system 300 is widely applicable to other analytical instruments besides mass spectroscopy. For example, a chromatography system such as high performance liquid chromatography (HPLC) could be used in place of mass spectrometer 340.

Given the plurality of spikes and analytes that may be present in the ionized mixture being analyzed by mass spectrometer 340 in IPMS system 300, a variety of mass spectrometer tunings may be used. For example, various settings such as capillary voltages, skimmer voltages, pulser voltages, and detector voltage levels comprise a mass spectrometer tuning. Each tuning is used to characterize a certain mass range. For example, one tuning may be used to characterize analytes of relatively low molecular weight whereas another tuning may be used to characterize analytes of higher molecular weight. The range of masses observable for a given tuning may be denoted as a mass window. The mass windows may be identified by an element within the window. For each sample being processed by mass spectrometer 340, a plurality of mass windows will typically be analyzed. As disclosed in U.S. application Ser. No. 11/329,536, filed Jan. 11, 2006, the contents of which are hereby incorporated by reference, one or more processors (not illustrated) in controller 320 that control IPMS system 300 may be configured with a “data analysis engine” (DAE). The DAE uses the identity of the process solution being sampled and the mass spectrometer tunings to identify peaks of interest in the resulting mass spectrums from mass spectrometer 340. The DAE performs a ratio measurement using the identified peaks to calculate the concentrations of the analytes.

The above-described embodiments of the present invention are merely meant to be illustrative and not limiting. It will thus be obvious to those skilled in the art that various changes and modifications may be made without departing from this invention in its broader aspects. Accordingly, the appended claims encompass all such changes and modifications as fall within the true spirit and scope of this invention.

What is claimed is:

1. A method of analyzing an organic additive in the presence of an acidic metal plating matrix in a metal plating solution, comprising:
providing a sample from the metal plating solution;
activating a metal-complexing resin with an acid to provide an activated metal-complexing resin;
eluting the sample through the activated metal-complexing resin to form a treated sample in which a concentration of the acidic metal plating matrix is reduced; and
determining a concentration of an organic additive in the metal plating solution by analyzing the treated sample.

2. The method of claim 1, wherein the acid is a weak acid.

3. The method of claim 1, wherein providing the sample comprising diluting a portion of the metal plating solution.

4. The method of claim 2, wherein the metal-complexing resin comprises a bis-picoylamine-containing chelation resin, and wherein the weak acid is acetic acid.

5. The method of claim 4, wherein the acetic acid comprises 0.5 M acetic acid.

6. The method of claim 3, further comprising:
(a) mixing the sample with a spike to allow equilibrium to occur therebetween;
(b) ionizing the equilibrated sample and spike in an atmospheric pressure ionizer (API) to produce ions;
(c) processing the ions in a mass spectrometer to provide a ratio response; and
(d) determining the concentration of the organic additive in the sample using the ratio response.

7. The method of claim 6, further comprising:
(e) cyclically repeating acts (a) through (d) under machine control to automatically monitor the concentration of the organic additive in the metal plating solution over time.

8. The method of claim 1, further comprising:
regenerating the metal-complexing resin with a basic solution.

9. The method of claim 8, wherein the basic solution is aqueous ammonium hydroxide.

10. An analytical apparatus, comprising:
a sample extraction module operable to extract a sample of known volume from a metal plating solution having an acidic metal plating matrix;
a sample dilution module operable to dilute the sample to provide a diluted sample;
a mixer operable to mix the diluted sample with a spike to form a spiked sample,
a column packed with metal-complexing resin to reduce a concentration of the acidid metal plating matrix in the spiked sample to provide a treated sample;
an atmospheric pressure ionizer operable to ionize the treated sample to produce ions;
a mass spectrometer operable to process the ions to provide a ratio response; and
a control system operable to control a cyclic extraction of samples, dilution of the samples, spiking of the diluted samples, treatment of the spiked diluted samples, ionization of the treated samples, processing of the ions to provide ratio responses, and processing of the ratio responses to characterize the concentration of an organic additive in the metal plating solution over time.

11. The analytical apparatus of claim 10, wherein the metal-complexing resin comprises a bis-picoylamine-containing chelation resin.

12. The analytical apparatus of claim 11, wherein the apparatus is operable to activate the bis-picoylamine-containing chelation resin with a weak acid prior to the treatment of the spiked sample.

13. The analytical apparatus of claim 12, wherein the apparatus is further operable to regenerate the bis-picoylamine-containing chelation resin with a basic solution after treatment of the spiked sample.

14. The analytical apparatus of claim 13, wherein the weak acid comprises acetic acid and the basic solution comprises ammonium hydroxide.

15. The analytical apparatus of claim 12, wherein the atmospheric pressure ionizer is an electrospray ionizer.
